(19) United States
(12) Patent Application Publication
(10) Pub. No.: US 2025/0131686 A1
(43) Pub. Date: Apr. 24, 2025

(54) IMAGE CONTOURING METHOD AND SYSTEM, AND COMPUTER-READABLE STORAGE MEDIUM

(71) Applicant: Shanghai United Imaging Healthcare Co., Ltd., Shanghai (CN)

(72) Inventors: Yida He, Shanghai (CN); Weiqi Xiong, Shanghai (CN); Hongcheng Yang, Shanghai (CN)

(21) Appl. No.: 18/925,231

(22) Filed: Oct. 24, 2024

(30) Foreign Application Priority Data
Oct. 24, 2023 (CN) 202311386851.X

(51) Int. Cl. G06V 10/46; G06T 7/30; G06T 11/60; G06V 10/25

(52) U.S. Cl. CPC G06V 10/46; G06T 7/30; G06T 11/60; G06V 10/25

(57) ABSTRACT

The embodiments of the present specification provide an image contouring method and system, and a computer-readable storage medium. The method includes: obtaining a three-dimensional image, the three-dimensional image including a plurality of slice images arranged in sequence; obtaining reference contouring information in a first slice image among the plurality of slice images; and determining, based on the reference contouring information, a target contouring in a second slice image among the plurality of slice images, the second slice image being adjacent to the first slice image.

### CROSS-REFERENCE TO RELATED APPLICATIONS

This application claims priority to Chinese patent application No. 202311386851.X, filed on Oct. 24, 2023, the content of which is hereby incorporated by reference in its entirety.

### TECHNICAL FIELD

The present disclosure relates to the field of image processing technologies, and in particular, to an image contouring method and system, and a computer-readable storage medium.

### BACKGROUND

Target volume contouring is an important part of a radiotherapy process for patients. Accurately contouring a contour of target volume is a prerequisite and guarantee for achieving precise radiotherapy. Since a contouring for target volume of tumor requires not only professional anatomical expertise but also further reference to pathological information of the patient, the contouring work is generally completed by physicians based on their experience in clinical practice. It is often tedious and time-consuming for the physicians to manually contour the target volume and organs at risk (OARs) of the patient.

With the advancement of medical technology and the development of artificial intelligence, the physicians can complete the contouring of the target volume of tumor with the assistance of the introduced automatic target volume segmentation algorithms of artificial intelligence, which reduces the workload of the physicians to a certain extent. However, in actual applications, the segmentation results obtained using artificial intelligence algorithms may still require further modification, supplementation or correction. Especially when there is a large number of image layers, manual fine corrections are required one by one, which takes a lot of time and effort.

Therefore, an image contouring method and system, and a computer-readable storage medium are provided, which can achieve automatic and adaptive adjustment of contourings of other image layers when a user contours a certain image layer or modifies a contouring of the certain image layer, thereby improving the accuracy of contouring while reducing manual operations.

### SUMMARY

One of embodiments of the present specification provides an image contouring method, which includes: obtaining a plurality of slice images; obtaining reference contouring information in a first slice image among the plurality of slice images; and determining, based on the reference contouring information, a target contouring in a second slice image among the plurality of slice images.

In an embodiment, the plurality of slice images are arranged in sequence, and the second slice image is adjacent to the first slice image.

In an embodiment, the reference contouring information includes a prior constraint for defining the target contouring.

In an embodiment, the reference contouring information includes contouring information for at least one region of interest.

In an embodiment, the reference contouring information further includes contouring information for an associated region around the at least one region of interest.

In an embodiment, obtaining the reference contouring information in the first slice image among the plurality of slice images includes:

generating initial contouring information in the first slice image among the plurality of slice images; the initial contouring information containing an initial contouring of at least one region of interest in the first slice image;

obtaining, based on the initial contouring information, the reference contouring information in the first slice image.

In an embodiment, the initial contouring information is determined based on manual contouring, or the initial contouring information is determined based on a preset segmentation algorithm or Artificial Intelligence (AI).

In an embodiment, determining, based on the reference contouring information, the target contouring in the second slice image among the plurality of slice images includes: modifying, by using contouring modification information determined based on the reference contouring information, an initial contouring in the second slice image to determine the target contouring.

In an embodiment, the contouring modification information reflects a difference between the initial contouring and a modified contouring of the first slice image.

In an embodiment, the initial contouring is represented by a first contouring mask and the modified contouring is represented by a second contouring mask; and the contouring modification information includes a contouring modification mask determined by obtaining a difference between the first contouring mask and the second contouring mask.

In an embodiment, modifying, by using the contouring modification information determined based on the reference contouring information, the initial contouring in the second slice image includes: registering the first slice image with the second slice image to determine a deformation field between the first slice image and the second slice image; and modifying, based on the deformation field and the contouring modification information, the initial contouring.

In an embodiment, the first slice image and the second slice image are each includes one or more regions of interest with associated region, and registering the first slice image with the second slice image to determine the deformation field between the first slice image and the second slice image includes: for each region of interest with associated region, processing, based on the initial contouring or a modified contouring of the region of interest with associated region in the first slice image, the first slice image to obtain a first reference image; for each region of interest with associated region, processing, based on the initial contouring or a modified contouring of the region of interest with associated region in the second slice image, the second slice image to obtain a second reference image; constructing, based on the first reference image and the second reference image of each region of interest with associated region, a registration objective function, and determining, by solving the registration objective function, the deformation field.

In an embodiment, modifying, by using contouring modification information determined based on the reference contouring information, an initial contouring in the second slice image to determine the target contouring comprises: determining modified part of contouring in the first slice image as the contouring modification information of the first slice image; and modifying corresponding part of the initial contouring in the second slice image corresponding to the modified part to determine the target contouring of the second slice image.

In an embodiment, modifying, by using contouring modification information determined based on the reference contouring information, an initial contouring in the second slice image to determine the target contouring includes: determining modified pixel points based on the contouring modification information of the first slice image; modifying corresponding pixel points in the second slice image corresponding to the modified pixel points to determine the target contouring of the second slice image.

In an embodiment, determining, based on the reference contouring information, the target contouring in the second slice image among the plurality of slice images includes: determining, by using reference contoured contour information determined based on the reference contouring information, the target contouring.

In an embodiment, determining, by using the reference contoured contour information determined based on the reference contouring information, the target contouring includes: registering the first slice image with the second slice image to determine a deformation field between the first slice image and the second slice image; and determining, based on the deformation field and the reference contoured contour information, the target contouring.

In an embodiment, determining, by using reference contoured contour information determined based on the reference contouring information, the target contouring includes: determining based on the reference contoured contour information of the first slice image reference, contour pixel points involved in a global deformation mapping operation; determining target contour pixel points corresponding to the reference contour pixel points in the second slice image, based on the deformation field; and modifying the target contour pixel points in the second slice image to determine the target contouring of the second slice image.

In an embodiment, the method further includes: performing one or more rounds of contouring operations, each round of contouring operation includes: determining, based on contouring information in the second slice image in a current round of contouring operation, contouring information in an adjacent slice image of the second slice image; the contouring information includes an initial contouring or a target contouring; determining whether a termination condition is met; and designating, in response to the termination condition being not met, the adjacent slice image as a second slice image in a next round of contouring operation, and performing the next round of contouring operation; or stopping, in response to the termination condition being met, the one or more rounds of contouring operations.

In an embodiment, the method further includes: generating, based on the first slice image and the second slice images after the contouring operations, a treatment plan for a target volume.

One of embodiments of the present specification provides an image contouring system, which includes a storage device and a processing device, the storage device being stored with computer instructions, which, when executed by the processing device, performs steps to: obtain a plurality of slice images; obtain reference contouring information in a first slice image among the plurality of slice images; and determine, based on the reference contouring information, a target contouring in a second slice image among the plurality of slice images.

In an embodiment, the system further includes a scanning device, the scanning device being configured to obtain a three-dimensional image of scanned object, and transmit the three-dimensional image to the processing device for the processing device to perform contouring processes on the three-dimensional image.

One of embodiments of the present specification provides a non-transitory computer-readable storage medium storing instructions therein, wherein the instructions, when executed by a processor, cause the processor to implement steps to: obtain a plurality of slice images; obtain reference contouring information in a first slice image among the plurality of slice images; and determine, based on the reference contouring information, a target contouring in a second slice image among the plurality of slice images.

One of embodiments of the present specification provides an image contouring method, including: obtaining a three-dimensional image, the three-dimensional image including a plurality of slice images arranged in sequence; obtaining reference contouring information in a first slice image among the plurality of slice images; and determining, based on the reference contouring information, a target contouring in a second slice image among the plurality of slice images, the second slice image being adjacent to the first slice image.

One of embodiments of the present specification provides an image contouring system, including: an image data obtaining module configured to obtain a three-dimensional image, the three-dimensional image including a plurality of slice images arranged in sequence; a reference contouring determination module configured to obtain reference contouring information in a first slice image among the plurality of slice images; and a target contouring determination module configured to determine, based on the reference contouring information, a target contouring in a second slice image among the plurality of slice images, the second slice image being adjacent to the first slice image.

One of embodiments of the present specification provides a computer-readable storage medium storing an instruction therein. The instruction, when executed by a processor, causes the processor to implement the above image contouring method.

### BRIEF DESCRIPTION OF THE DRAWINGS

The present specification will be further illustrated by means of exemplary embodiments, which will be described in detail through the accompanying drawings. These embodiments are not limitative, and in these embodiments, like numbers represent like structures.

FIG. 1 is a schematic diagram illustrating an application scenario of an image contouring system according to some embodiments of the present specification.

FIG. 2 is a schematic diagram illustrating modules of an image contouring system according to some embodiments of the present specification.

FIG. 3 is an exemplary flow diagram illustrating an image contouring method according to some embodiments of the present specification.

FIG. 4 is an exemplary flow diagram illustrating a method for determining a target contouring of a second slice image according to some embodiments of the present specification.

FIG. 5 is an exemplary flow diagram illustrating a method for registering a first slice image with a second slice image according to some embodiments of the present specification.

FIG. 6 is an exemplary flow diagram illustrating one or more rounds of contouring operations according to some embodiments of the present specification.

FIG. 7 is an exemplary flow diagram illustrating another image contouring method according to some embodiments of the present specification.

FIG. 8 is an exemplary flow diagram illustrating yet another image contouring method according to some embodiments of the present specification.

### DETAILED DESCRIPTION OF THE EMBODIMENTS

In order to describe the technical solutions of the embodiments of the present specification more clearly, the accompanying drawings required for describing the embodiments will be briefly introduced as follows. Apparently, the accompanying drawings, in the following description, illustrate merely some examples or embodiments of the present specification, for a person of ordinary skill in the art, the present specification can be applied to other similar scenarios according to these accompanying drawings without making any creative efforts. Unless otherwise apparent from the context or indicated otherwise, the same reference numerals in the figures represent the same structure or operation.

It should be understood that the “system”, “device”, “unit” and/or “module” used herein is a method for distinguishing different components, elements, parts, portions, or assemblies at different levels. However, if other words can achieve the same purpose, the words may be replaced by other expressions.

Unless the context clearly indicates an exception, the words “a”, “an”, “one kind” and/or “the” do not specifically refer to the singular but may include the plural. Generally, the terms “include” and “comprises” only indicate the inclusion of explicitly identified steps and elements, and these steps and elements do not constitute an exclusive list, and the method or device may also include other steps or elements.

In the present specification, flow diagrams are used to explain operations performed by the system according to the embodiments of this specification. It should be understood that the preceding or following operations do not have to be performed precisely in order. Instead, the steps may be processed in reverse order or simultaneously. Meanwhile, other operations can be added to these processes, or one or more operations can be removed from these processes.

The present specification is mainly about contouring a region of interest. A contouring operation may include determining contour information of the region of interest in a medical image, or may include drawing a contour of the region of interest in the medical image. Accordingly, a contouring obtained by the contouring operation may be represented by the contour information (such as location information of contour points), or by image elements (such as lines used to represent the contours). The region of interest refers to a region in the image that needs to be contoured, such as target volume. The region of interest corresponds to actual organs and/or tissues on a surface of or inside the body of a target object, and these tissues and/or organs have corresponding visual representations in the image. For illustrative purposes, in this specification, the region of interest is used to collectively refer to a region in an image and tissues and/or organs in physical space corresponding to the region.

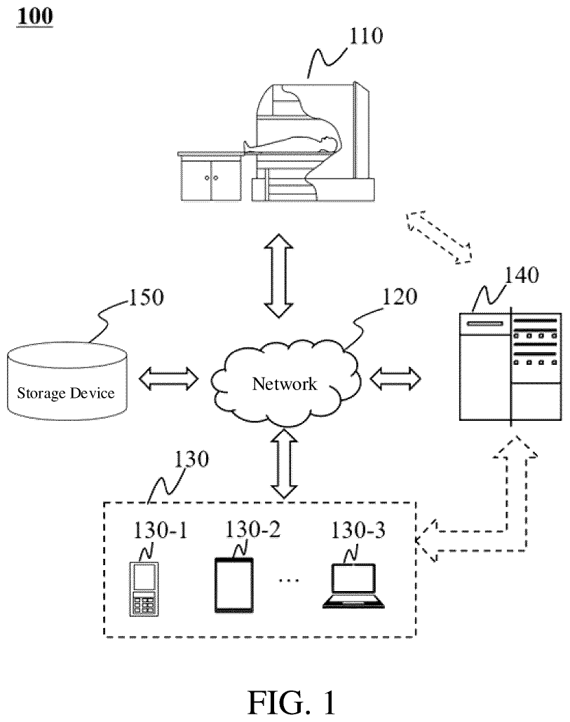

FIG. 1 is a schematic diagram illustrating an application scenario of an image contouring system according to some embodiments of the present specification.

As shown in FIG. 1, the application scenario 100 of the image contouring system may include a medical scanning device 110, a network 120, a terminal 130, a processing device 140, and a storage device 150. The components in the application scenario 100 may be connected in various ways. By way of example only, as shown in FIG. 1, the medical scanning device 110 may be connected to the processing device 140 via the network 120. The medical scanning device 110 may be directly connected to the processing device 140 (as indicated by the double-headed arrow in the dashed line connecting the medical scanning device 110 and the processing device 140. The storage device 150 may be connected to processing device 140 directly or through the network 120.

The medical scanning device 110 may scan a scanned object and/or generate data regarding the scanned object. Exemplarily, the medical scanning device 110 may include a CT device, a PET/CT device, an MRI/CT device, or the like. In this specification, the scanned object may also be referred to as a scanning object, a target object, an object, or a detected object. In some embodiments, the target object may be a patient, an animal, or the like. When the target object needs to be scanned, the medical scanning device 110 can obtain a medical image (e.g. a three-dimensional image including a plurality of slice images arranged in sequence) corresponding to the target object.

The network 120 may include any suitable network that facilitates the exchange of information and/or data for the application scenario 100. In some embodiments, one or more components of the application scenario 100 (e.g., the medical scanning device 110, the terminal 130, the processing device 140, or the storage device 150) may communicate information and/or data with one or more other components of the application scenario 100 via the network 120. For example, the processing device 140 may obtain a medical image (e.g. a three-dimensional image including a plurality of slice images arranged in sequence) of a scanning object from the medical scanning device 110 via the network 120. In some embodiments, the network 120 may be any one or more of a wired network or a wireless network. In some embodiments, the network may be of various topologies such as point-to-point, shared, centralized, etc., or a combination of multiple topologies.

The terminal 130 may include a mobile device 130-1, a tablet computer 130-2, a laptop computer 130-3, etc., or any combination thereof. In some embodiments, the terminal 130 may interact with other components in the application scenario 100 through the network 120. For example, the terminal 130 may receive data such as the medical image (e.g. a three-dimensional image including a plurality of slice images arranged in sequence) sent by the medical scanning device 110. In some embodiments, the terminal 130 may receive information and/or instructions input by a user (e.g., a user of the medical scanning device 110, such as a doctor), and send the received information and/or instructions to the medical scanning device 110 or the processing device 140 via the network 120. For example, a doctor can modify an initial contouring in a certain slice image through the terminal 130.

In some embodiments, the terminal 130 may include various applications for processing (e.g., contouring) medical image data.

The processing device 140 may process data and/or information obtained from the medical scanning device 110, the terminal 130, and/or the storage device 150. For example, the processing device 140 may obtain a medical image of a scanned person. In some embodiments, the processing device 140 can process the medical image. For example, the processing device 140 can generate a contouring of the region of interest in whole or a part of the medical image. In some embodiments, the processing device 140 may obtain data and/or instructions from the terminal 130, and perform corresponding processing. For example, it can modify contourings in one or more images according to a contouring modification instruction.

In some embodiments, the processing device 140 may be a single server or a group of servers. The group of servers may be centralized or distributed. In some embodiments, the processing device 140 may be local or remote. The processing device 140 may be directly connected to the medical scanning device 110, the terminal 130, and the storage device 150 to access stored or obtained information and/or data. In some embodiments, the processing device 140 may be implemented on a cloud platform. By way of example only, the cloud platform may include a private cloud, a public cloud, a hybrid cloud, a community cloud, a distributed cloud, an internal cloud, a multi-layer cloud, or the like, or any combination thereof.

The storage device 150 can store data and/or instructions. In some embodiments, the storage device 150 can store data obtained from the medical scanning device 110, the terminal 130, and/or the processing device 140. For example, the storage device 150 can store medical images obtained by a user operating scanning device. In some embodiments, the storage device 150 can store data and/or instructions for the processing device 140 to execute the exemplary methods described in this specification. For example, the storage device 150 can store instructions for the processing device 140 to execute the methods shown in respective flow diagrams. In some embodiments, the storage device 150 may include a mass storage device, a removable storage device, a volatile read-write memory, a read-only memory (ROM), etc., or any combination thereof. In some embodiments, the storage device 150 may be implemented on the cloud platform. In some embodiments, the storage device 150 may be a part of the processing device 140.

The above description is for illustrative purposes only, and actual application scenarios may vary.

It should be noted that the application scenario 100 is provided for illustration purposes only, and is not intended to limit the scope of the present disclosure. It is obvious to those skilled in the art that various modifications or changes can be made based on the description of the present specification. However, these changes and modifications will not depart from the scope of the present disclosure.

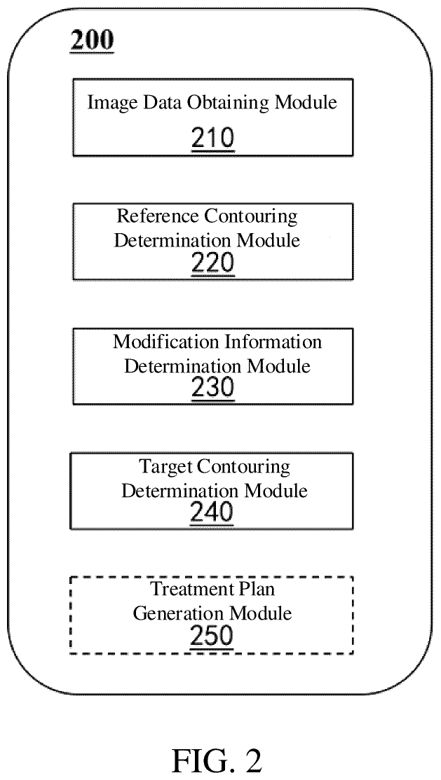

FIG. 2 is a schematic diagram illustrating modules of an image contouring system according to some embodiments of the present specification.

As shown in FIG. 2, the image contouring system 200 (hereinafter referred to as a contouring system) may include an image data obtaining module 210, a reference contouring determination module 220, a modification information determination module 230, and a target contouring determination module 240. In some embodiments, the contouring system may be implemented by the processing device 140 shown in FIG. 1.

The image data obtaining module 210 may be configured to obtain a three-dimensional image. The three-dimensional image includes a plurality of slice images arranged in sequence.

The reference contouring determination module 220 may be configured to obtain reference contouring information in a first slice image among the plurality of slice images.

In some embodiments, the reference contouring information includes contouring information for at least one region of interest, and contouring information for an associated region around the at least one region of interest.

In some embodiments, the reference contouring determination module 220 may be further configured to generate an initial contouring of at least one region of interest in each of the slice images to obtain the reference contouring information in the first slice image.

The modification information determination module 230 may be configured to determine contouring modification information based on a modification instruction for the initial contouring in the first slice image among the plurality of slice images.

The target contouring determination module 240 may be configured to determine, based on the reference contouring information, a target contouring in a second slice image among the plurality of slice images. The second slice image is adjacent to the first slice image.

In some embodiments, the target contouring determination module 240 may be further configured to determine, by using reference contoured contour information determined based on the reference contouring information, the target contouring.

In some embodiments, the target contouring determination module 240 may be configured to modify, by using contouring modification information determined based on the reference contouring information, an initial contouring in the second slice image to determine the target contouring.

In some embodiments, the target contouring determination module 240 may be further configured to: register the first slice image with the second slice image to determine a deformation field between the first slice image and the second slice image; and modify, based on the deformation field and the contouring modification information, the initial contouring.

In some embodiments, the target contouring determination module 240 may be further configured to: for each region of interest, process, based on the initial contouring of the region of interest in the first slice image, the first slice image to obtain a first reference image; process, based on the initial contouring of the region of interest in the second slice image, the second slice image to obtain a second reference image; and construct, based on the first reference image and the second reference image of the each region of interest, a registration objective function, and determine the deformation field by solving the registration objective function.

In some embodiments, the target contouring determination module 240 may be further configured to perform, based on the initial contouring of the region of interest in the first slice image, a contrast enhancement processing on the first slice image to increase a contrast ratio between a contoured region and an external region in the first slice image.

In some embodiments, the target contouring determination module 240 may be further configured to perform one or more rounds of contouring operations. Each round of contouring operation includes: determining, based on contouring information in the second slice image in a current round of contouring operation, contouring information in an adjacent slice image of the second slice image; determining whether a termination condition is met; designating, in response to the termination condition being not met, the adjacent slice image as a second slice image in a next round of contouring operation and performing the next round of contouring operation; and stopping, in response to the termination condition being met, the one or more rounds of contouring operations.

In some embodiments, the target contouring determination module 240 may be further configured to: determine a difference between an initial contouring in the adjacent slice image and a target contouring in the adjacent slice image; determine, in response to the difference being greater than a preset difference threshold, that the termination condition is not met; determine, in response to the difference being less than or equal to the preset difference threshold, that the termination condition is met.

In some embodiments, the image contouring system 200 may further include a treatment plan generation module 250.

The treatment plan generation module 250 may be configured to generate a treatment plan for the target volume based on the target contouring in the slice image that has been modified the contouring and/or the initial contouring in the slice image that has not been modified the contouring.

It should be noted that the functions described in the above modules can be achieved by the image contouring system 200 after executing computer instructions (program codes). In some embodiments, the contouring system 200 may include one or more processing devices (e.g., the processing device 140 shown in FIG. 1). In some embodiments, the contouring system 200 may include a corresponding storage medium (e.g., a memory) or be signal-connected to an external storage medium. The storage medium can store instructions for executing the image contouring method disclosed in the present specification. When the processing device reads these instructions, the processing device may be configured to execute an image contouring method.

It should be noted that the above description of the contouring system 200 and the corresponding modules is for convenience of description only, and does not limit the present specification to the scope of the embodiments described. It is understandable that, for those skilled in the art, after understanding the principle of the system, it is possible to arbitrarily combine the modules or form a subsystem connected with other modules without deviating from the principle. For example, the reference contouring determination module 220, the modification information determination module 230, and the target contouring determination module 240 may be different modules in the system, or a single module that implements the functions of two or more of the above modules. For example, all modules may share one storage module, or each module may have its own storage module. Such variations are all within the protection scope of the present specification.

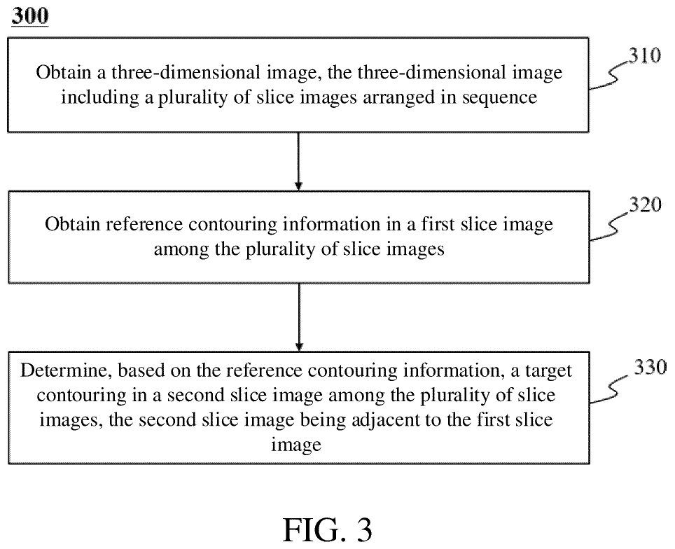

FIG. 3 is an exemplary flow diagram illustrating an image contouring method according to some embodiments of the present specification.

In some embodiments, process 300 can be performed by a contouring system (e.g., the image contouring system 200 shown in FIG. 2). As shown in FIG. 3, the process 300 includes the following steps.

In step 310, a three-dimensional image is obtained. The three-dimensional image includes a plurality of slice images arranged in sequence.

The three-dimensional image may include, but is not limited to, a three-dimensional medical image. For example, it may be a three-dimensional medical image of a target object in a medical scenario (such as radiotherapy). The following will take the three-dimensional medical image as an example for illustration. The target object may be any object whose three-dimensional medical image needs to be contoured. In some embodiments, the target object may be an object requiring radiotherapy, such as a cancer patient. In some embodiments, the target object needs to receive fractionated radiotherapy using therapeutic radiation emitted by a radiotherapy device because of lesions (e.g., tumors) in certain parts of the body.

The three-dimensional medical image may be obtained based on a medical imaging equipment such as an ultrasound acquisition device, a CT scanner, a PET scanner, an MRI scanner, or the like.

In some embodiments, the three-dimensional medical image may be presented on a terminal device (e.g., the terminal 130), allowing a user (e.g., a doctor, a contouring operator, etc.) to switch between different perspectives to observe and analyze the three-dimensional medical image.

The slice image refers to a two-dimensional image layer in the three-dimensional medical image. The slice image may include, but is not limited to, a transverse plane image, a coronal plane image, or a sagittal plane image.

In some embodiments, the plurality of slice images may be arranged in sequence according to their locations in the three-dimensional medical image. For example, the three-dimensional medical image corresponds to a three-dimensional image coordinate system (including an x-axis, a y-axis, and a z-axis). Assuming that the xy plane corresponds to a cross-section of the human body, then a plurality of cross-sectional images can be determined, and these cross-sectional images are arranged in order from small to large according to the corresponding z-coordinate.

It should be noted that the plurality of slice images may be presented on the terminal device (such as a display interface) through a plurality of display windows arranged in sequence, or may also be presented through one display window, and the user may switch the displayed slice images through an input device (such as switching up and down on a keyboard, scrolling a mouse, etc.).

In step 320, reference contouring information in a first slice image among the plurality of slice images is obtained.

The reference contouring information refers to contouring information used to provide reference or guidance for contourings of a part and/or all of the slice images in the three-dimensional image. The reference contouring information may be overall contouring information in a slice image, or local contouring modification information in the existing overall contouring information. For example, in the case the target contouring is confirmed by the user after the user has made modification to the first slice image, that is, the target contouring is the modified contouring confirmed by the user, then the reference contouring information may include the contouring modification information corresponding to the user's modification, and/or may include the confirmed modified contouring of the first slice image. In the case that the target contouring is confirmed by the user without making modification to the first slice image, that is, the target contouring is the original contouring confirmed by the user, then the reference contouring information may include the confirmed initial contouring of the first slice image.

In some embodiments, the reference contouring information may include contouring information for at least one region of interest, and contouring information for an associated region around the at least one region of interest.

The first slice image refers to a slice image including the reference contouring information. In some embodiments, the first slice image refers to a slice image on which the user is to perform a contouring modification. The first slice image may be a slice image specified by the user. For example, the first slice image may be one or a combination of a 1st to nth slice images of the plurality of slice images arranged in sequence (such as a sequence consisting of n slice images). It should be understood that there may be only a part of the plurality of slice images includes the region of interest, and the first slice image and the second slice image described below each refer to a slice image that includes the region of interest.

In some embodiments, the contouring system may select a representative image from the plurality of slice images, and recommend it to the user for review or modification. For example, the representative image may be a slice image containing the largest area of the region of interest in the slice image sequence. When the user performs a contouring modification on the representative image, the representative image is regarded as the first slice image.

The region of interest may refer to a region that needs to be contoured in the image. For example, the region of interest may include a region related to tumor treatment (such as X-ray irradiation therapy, radioactive particle implantation therapy, etc.). The region of interest may include a region of target volume of tumor. The associated region around the region of interest may be a region of organ at risk around the region of interest (e.g., within a preset distance range). For example, the associated region may be a region of a normal organ near the target volume that may be accidentally injured due to radiation during radiotherapy, or may be other regions of non-irradiated tissues around the target volume that may be affected. By way of example only, for a cervical cancer patient, the region of interest and the associated region around the region of interest may include a region of target volume of tumor of a cervical cancer, and a region of at least one or a combination of OARs such as the levator ani muscle, rectum, and pubis. For ease of description, the region of interest and the corresponding associated region described below may be collectively referred to as the region of interest.

In some embodiments, the contouring system may generate, for each of the slice images, an initial contouring of at least one region of interest in the slice image. For example, the contouring system may generate initial contouring information of the first slice image to obtain the reference contouring information in the first slice image.

The initial contouring refers to a preliminarily determined contour (also called a boundary) of at least one region of interest in the slice image. The initial contouring may be represented by information indicating the position of the boundary line of the contour, for example, by coordinates of contour points. The initial contouring may also be represented by a first contouring mask. The contouring mask in this specification is an image that can represent the contour points, for example, a image in which pixel values of the contour points are each being a first preset value (such as 1), and pixel values of other pixel points are each being a second preset value (such as 0).

In some embodiments, the initial contouring may be determined based on manual contouring (e.g. manual drawing). For example, a user (such as a doctor) may draw, by using software or an application (such as 3D Slicer) and through an input device (such as a mouse or a keyboard), contours of the target volume of tumor and OARs in the slice image to obtain the initial contouring of the slice image.

In some embodiments, the initial contouring may be determined based on a preset segmentation algorithm. For example, the contouring system may segment the slice image by using various segmentation algorithms, or artificial intelligence technologies (such as trained deep learning models) to determine the contours of the target volume and OARs in the slice image, thereby obtaining the initial contouring in the slice image.

In some embodiments of the present specification, the plurality of slice images may be automatically segmented in batches through a preset segmentation algorithm to obtain initial contourings of the slice images, thereby reducing the overall workload and time required for the contouring.

In step 330, a target contouring in a second slice image among the plurality of slice images is determined based on the reference contouring information. The second slice image is adjacent to the first slice image.

In some embodiments, the second slice image may be determined based on a preset configuration. For example, the second slice image may be one or more slice images specified by the user.

In some embodiments, the second slice image may be a slice image that is adjacent to the first slice image in the slice image sequence and includes the region of interest. The slice image sequence is consisted of a plurality of slice images arranged in sequence. For example, the plurality of slice images may be a plurality of 2D images obtained by taking cross-sectional images at an equal thickness interval from a three-dimensional image of an imaged object and then arranging the taken cross-sectional images in positional sequence, or a plurality of 2D images obtained by taking cross-sectional images at an equal time interval from an imaged object and then arranging the taken cross-sectional images in time sequence. Exemplarily, the first slice image is the mth slice image in the slice image sequence, and the second slice image is the (m-1)th (previous) and/or (m+1)th (next) slice image. Exemplarily, the first slice image and the second slice image that are adjacent to each other may be two images which having a highest similarity with each other or which having a similarity higher than a preset similarity threshold, among the plurality of slice images.

The target contouring refers to contouring information generated in the second slice image according to the reference contouring information of the first slice image.

In some embodiments, the contouring system may determine the target contouring based on the local contouring modification information of the reference contouring information.

In some embodiments, the contouring system may determine contouring modification information based on a modification instruction to the initial contouring in the first slice image of the plurality of slice images. As mentioned above, initial contouring can be determined manually based on manual drawing or automatically based on a preset segmentation algorithm. Then, the contouring image generated based on the initial contouring can be displayed on the display of the terminal device for the user to check and confirm whether the contouring image meets the use needs. For example, in cases where the machine performs a segmentation algorithm to generate an initial contouring, the resulting initial contouring may not be accurate enough, in which case the user may need to modify the generated initial contouring to make it more accurate. For another example, for the second slice image, the contouring of the second slice image determined automatically by the system based on the reference contouring information in the first slice image may not be accurate enough, so the user may also need to modify the contouring of the second slice image determined by the system to make it more accurate, and then the user may confirm the modified contouring as the target contouring of the second slice image.

In some embodiments, for the initial contouring of one or more slice images, the user may determine whether the initial contouring needs to be modified according to actual requirements. Exemplarily, the user may modify the initial contouring according to factors such as a medical history (such as a record of recurrence, a record of complication symptoms, etc.) of the patient, the coverage or envelopment of the tumor by the initial contouring, and an effect of the dose on OARs.

In some embodiments, the contouring system may obtain the modification instruction for the initial contouring from the user, and adjust the initial contouring accordingly. The modification instruction may be input through the input device (such as a keyboard, a mouse, or a microphone), and configured to expand, shrink or fine-tune the contour of the initial contouring. The initial contouring modified by the user is referred to as a modified contouring hereinafter. The modified contouring may also be represented by coordinates of contour points and/or a second contouring mask.

The contouring modification information may reflect a difference between the initial contouring and the modified contouring of the first slice image. In other words, the contouring modification information may reflect what modifications the user has made to the initial contouring through the modification instruction. The modified contouring may have a boundary line of contour different from that of the initial contouring. Correspondingly, the contouring modification information may include word or image representations illustrating deleting, adding or modification to the boundary line of contour. For example, the contouring modification information may reflect changes in the contour, shape, size, area, etc. of the region of interest between the initial contouring and the modified contouring. For another example, the contouring modification information may reflect a difference between the contour points in the initial contouring and the contour points in the modified contouring.

In some embodiments, the contouring modification information may be determined based on the contour points in the initial contouring and the contour points in the modified contouring. For example, a contouring modification mask may be generated as the contouring modification information based on the contour points in the initial contouring and the contour points in the modified contouring. In the contouring modification mask, points with a pixel value of 1 are non-contour points in the initial contouring and are contour points in the modified contouring; points with a pixel value of −1 are contour points in the initial contouring and are non-contour points in the modified contouring; and points with a pixel value of 0 are contour points in both the initial contouring and the modified contouring, or are non-contour points in both the initial contouring and the modified contouring. The pixel points with pixel values of −1 and 1 in the contouring modification mask are the points modified by the user when performing contouring modification on the first slice image, and are referred to as modified pixel points hereinafter. In some embodiments, the contouring modification mask may be determined by obtaining a difference between the first contouring mask and the second contouring mask, for example, by subtracting the first contouring mask corresponding to the initial contouring from the second contouring mask corresponding to the modified contouring.

In some embodiments, the contouring system may modify an initial contouring in the second slice image by using the contouring modification information determined based on the reference contouring information to determine the target contouring.

Exemplarily, after a boundary of a local region of the target volume in the first slice image is expanded outward, the contouring system may modify the corresponding plurality of pixel points in the second slice image based on the contouring modification information corresponding to pixel points on the boundary of the local region (such as setting the pixel value of the pixel point to 1) to obtain a modified contour of the target volume in the second slice image, and the modified contour is the target contouring.

In some embodiments, when the initial contouring is generated, the initial contouring may be displayed on a display for user to check. When a modified contouring is generated, the modified contouring can be displayed on the display for the user to check. That is, the newly generated contouring (initial contouring or modified contouring) can be displayed for the user to check so that the user can confirm whether the current generated contouring as displayed meets the use needs.

In some embodiments, whether a current contouring (initial contouring or modified contouring) generated for a slice image can be determined as the final target contouring for this slice image, can be confirmed based on a confirmation instruction input by the user. In some embodiments, the user may input a confirmation instruction to the system when the current contouring in the confirmation system (for example, the latest initial contouring or modified contouring as displayed) meets the needs, and the system may take the current contouring confirmed by the user as the target contouring of the current slice image. For example, when an initial contouring is generated, the user may check whether the initial contouring meets the needs. If yes, the user may input a confirmation instruction to the system. If no, the user may input one or more modification instructions to modify the initial contouring for once or more times, until the initial contouring has been modified to meet the user's needs. When the initial contouring has been modified to meet the needs, the user may input a confirmation instruction to the system to confirm the currently modified contouring as the target contouring. Accordingly, when the system receives the confirmation instruction input by the user, if the current/latest contouring is the initial contouring, that is, the user confirms the initial contouring without modifying it, then the system may regard the initial contouring as the target contouring; when the system receives the confirmation instruction input by the user, if the current/latest contouring is a modified contouring, that is, the user has modified the initial contouring before confirming it, then system may regard the current modified contouring as the target contouring.

In some embodiments, the contouring system may use contouring modification information contained in the reference contouring information of a previous slice image (a first slice image or a second slice image) to modify an initial contouring of a latter slice image (a second slice image) to obtain a modified initial contouring of the latter slice image. Then the initial contouring of the latter slice image can be updated with the modified initial contouring. The updated initial contouring of the latter slice image may then be displayed, so that the user can confirm whether the updated initial contouring can be confirmed as a target contouring of the latter slice image. Thereafter, if the updated initial contouring of the latter slice image meets the use needs, the user may directly confirm the updated initial contouring as the target contouring of the latter slice image; if the updated initial contouring of the latter slice image fails to meet the use needs, the user may modify the updated initial contouring to obtain a modified contouring, and may confirm the modified contouring as the target contouring of the latter slice image when the modified contouring meets the use needs.

In some embodiments, the contouring system may use target contouring information (initial contouring information or modified contouring information) contained in the reference contouring information of a previous slice image (a first slice image or a second slice image), to map the object contouring of the previous slice image to the latter slice image (second slice image), so as to generate an initial contouring of the latter slice image. In this way, the generated initial contouring is updated as the initial contouring of the latter slice image. The updated initial contouring of the latter slice image may then be displayed, so that the user can confirm whether the updated initial contouring can be confirmed as a target contouring of the latter slice image. Thereafter, if the updated initial contouring of the latter slice image meets the use needs, the user may directly confirm the updated initial contouring as the target contouring of the latter slice image; if the updated initial contouring of the latter slice image fails to meet the use needs, the user may modify the updated initial contouring to obtain a modified contouring, and may confirm the modified contouring as the target contouring of the latter slice image when the modified contouring meets the use needs.

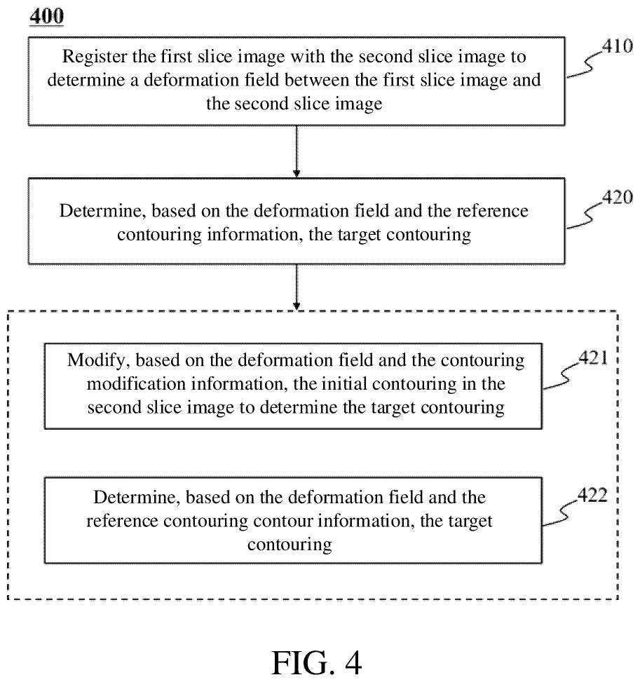

In some embodiments, the contouring system may determine the target contouring based on a deformation field between the first slice image and the second slice image. For relevant information about the deformation field, FIG. 4 and the corresponding description are referred.

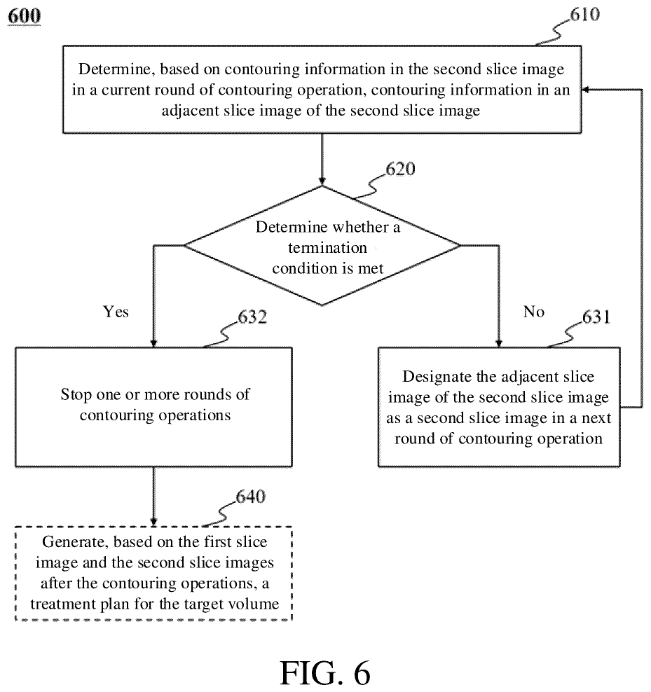

In some embodiments, the contouring system may further perform one or more rounds of contouring operations based on the initial contouring of the second slice image and the target contouring obtained after the modification thereof, until a termination condition is met. For relevant information about multiple rounds of contouring operations and the termination condition, FIG. 6 and the corresponding description are referred.

In some embodiments of the present specification, a preliminary contouring in each of the slice images can be quickly obtained through the preset segmentation algorithm, which provides a basis for subsequent contouring modification work and improves the efficiency of the overall contouring work. Furthermore, based on the deformation field and the contouring modification information of the first slice image, mapping of local contouring modification of the second slice image adjacent to the first slice image can be achieved, thereby improving the efficiency of contouring modification and making the contouring modification more in line with user expectations. In addition, based on the initial contouring and the target contouring in the second slice image, the plurality of image layers of the overall three-dimensional medical image can be automatically contoured and modified layer-by-layer through multiple rounds of contouring operations, which greatly improves the efficiency of contouring modification and reduces the contouring workload of the user.

FIG. 4 is an exemplary flow diagram illustrating a method for determining the target contouring of the second slice image according to some embodiments of the present specification.

In some embodiments, process 400 may be performed by the contouring system. As shown in FIG. 4, the process 400 includes the following steps.

In step 410, the first slice image is registered with the second slice image to determine a deformation field between the first slice image and the second slice image.

The deformation field may be used to determine pixel points (e.g., pixel points corresponding to the same physical point) in the first slice image and the second slice image that correspond to each other anatomically, and a spatial transformation relationship between corresponding pixel points. The spatial transformation relationship includes a displacement direction and displacement amount of the pixel point. For example, the deformation field from the first slice image to the second slice image can be used to translate one or more pixels of the first slice image in a specific direction, thereby achieving consistency in spatial location (such as anatomical structure) between these pixels and corresponding pixels in the second slice image. In some embodiments, the deformation field includes a deformation vector for each pixel point in the first slice image. For each pixel point in the first slice image, a corresponding pixel point in the second slice image can be determined based on location coordinates and the deformation vector of the pixel point.

In some embodiments, the contouring system may register the first slice image with the second slice image based on various registration algorithms to determine the deformation field between the first slice image and the second slice image. The various registration algorithms include but are not limited to grayscale-based matching algorithms, feature-based matching algorithms, and domain transformation-based matching algorithms.

In some embodiments, the contouring of the associated region (such as organs at risk) in the first slice image and the contouring of the associated region (such as organs at risk) in the second slice image may be used to perform the registration of the first slice image and the second slice image second slice image.

In some embodiments, the second slice image may be represented as a reference image in the registration process. The second slice image can be represented based on the following formula (1):

$$R ( x ) = R ( {x}_{1} , {x}_{2} , {x}_{3} ) \qquad Formula ( 1 )$$

where R (x) in formula (1) represents the second slice image, and x₁, x₂, and x₃ represent three-dimensional coordinate values in the second slice image.

The first slice image may be represented as a transformed image that needs to be spatially transformed in the registration process. The first slice image can be represented based on the following formula (2):

$$T ( x ) = T ( {x}_{1} , {x}_{2} , {x}_{3} ) \qquad Formula ( 2 )$$

where T (x) in formula (2) represents the first slice image, and x1, x2, and x3 represent three-dimensional coordinate values in the first slice image.

The deformation field U (x)=[u (x), v (x), w (x)] from the first slice image to the second slice image can be determined through registration. The deformation field can be implemented as shown in the following formula (3):

$$T ( x + U ( x ) ) \approx R ( x ) \qquad Formula ( 3 )$$

where u (x), v (x), w (x) in the deformation field U (x)=[u (x), v (x), w (x)] represent displacement amount of each pixel point in the first slice image on the three-dimensional coordinate axis, respectively. For each pixel point in the first slice image, a corresponding pixel point in the second slice image can be determined based on the coordinates of the pixel point, and u (x), v (x), and w (x).

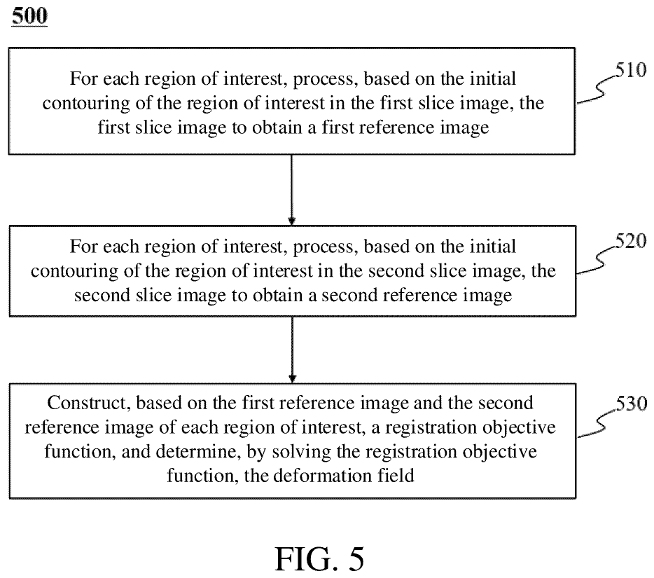

In some embodiments, the registration of the first slice image and the second slice image may be performed based on a reference contouring (such as the initial contouring or the contouring modified by the user) in the first slice image and the initial contouring in the second slice image. Meanwhile, considering the associated region (such as organs at risk) around the region of interest, the associated region is introduced as a prior constraint in the reference contouring information in the first slice image. The prior constraint can be used for defining the target contouring in the second slice image. By adding existing contouring or profile (such as contouring or profile of existing target volume and/or OARs) as a prior constraint in the registration, which can optimize the deformation field generated by the registration, the obtained deformation field can be made more accurate when there is a great difference between the images of two adjacent layers. For more details, FIG. 5 and the corresponding description are referred. Not limiting to the above examples, the prior constraint can include the size of the OARs, the boundaries between the target volume and OARs. As exemplary embodiments, the profile (or the contouring) of existing target volume and/or OARs can be used as prior constraint to define the target contouring in the second slice image in the following way: determining the target contouring in the second slice image such that the profile of the OARs do not overlap with said target contouring, or constructing a contouring optimization function for determining the target contouring by optimization using the boundaries of the profile of the OARs as a constraint.

In some embodiments, the deformation field may be determined by using a contouring (e.g., the initial contouring, the target contouring) of a region of interest and/or an organ at risk in the first image slice and a contouring (e.g., the initial contouring) of the region of interest and/or the organ at risk in the second image slice as a constraint. The target contouring of the second image slice may be determined based on the deformation field. For example, the deformation field may be determined by using a contouring (e.g., the initial contouring, the target contouring) of an organ at risk in the first image slice and a contouring (e.g., the initial contouring) of the organ at risk in the second image slice as the constraint.

In step 420, the target contouring is determined based on the deformation field and the reference contouring information.

In some embodiments, the contouring system may determine the target contouring according to step 421 or step 422 as shown below. In some embodiments, in the case that there is a modified contouring in the first slice image and an initial contouring in the second slice image, the step 421 can be performed. For example, if the first slice image has a modified contouring of the region of interest (such as target region), while the second slice image has a contouring of the associated region (such as organs at risk) and also has an initial contouring of the region of interest, the step 421 can be performed. In some embodiments, in the case that there is an initial contouring or modified contouring in the first slice image while no initial contouring in the second slice image, the step 422 can be performed. For example, if the first slice image has an initial contouring or a modified contouring of the region of interest (such as target region), while the second slice image has a contouring of the associated region (such as organs at risk) but do not have an initial contouring of the region of interest, the step 422 can be performed. For relevant information about the reference contouring information, FIG. 3 and the corresponding description are referred.

In step 421, the initial contouring in the second slice image is modified based on the deformation field and the contouring modification information to determine the target contouring in the second slice image.

In some embodiments, the contouring system may perform a contouring operation on the initial contouring in the second slice image adjacent to the first slice image based on the deformation field and the contouring modification information of the first slice image. In this case, the contouring operation may also be called a local deformation mapping operation.

In some embodiments, modifying, by using contouring modification information determined based on the reference contouring information, an initial contouring in the second slice image to determine the target contouring may include: determining modified part of contouring in the first slice image as the contouring modification information of the first slice image; and modifying corresponding part of the initial contouring in the second slice image corresponding to the modified part to determine the target contouring of the second slice image. The part of contouring may be represented by image features in the slice image, for example, pixel points, lines, curves, parameters, etc.

In some embodiments, a part of contouring that is modified in the first image slice may be determined. A corresponding part of contouring in the second image slice is modified, based on the deformation field between the first image slice and the second image slice, to determine the target contouring of the second slice image.

Exemplarily, the contouring system may determine modified pixel points involved in the contouring modification (such as pixel points with values of −1 and 1 in the contouring modification mask) based on the contouring modification mask of the first slice image, determine pixel points in the second slice image corresponding to the aforementioned modified pixel points based on the deformation field or by using AI recognition, etc., and modify these pixel points in the second contouring mask (such as modifying pixel values of these pixel points), thereby obtaining an updated contouring mask for the second slice image. According to the updated contouring mask of the second slice image, the target contouring of the second slice image can be determined. In this way, the local deformation mapping operation can be completed.

In some embodiments of the present specification, the contouring modification of the first slice image can be mapped to the second slice image in a local mapping manner through the deformation field and the contouring modification information. In the local mapping, it is only necessary to determine the pixel points corresponding to the modified pixel points of the first slice image in the second slice image, and modify these pixel points. Compared with mapping the modified contouring in the first slice image to the second slice image as a whole, the local mapping manner can save computing resources and improve the efficiency of automatic contouring modification. Meanwhile, the local mapping manner can also avoid modifying pixel points not involved in the contouring modification, thereby improving the accuracy of mapping of contouring modification.

In step 422, the target contouring in the second slice image is determined based on the deformation field and the reference contoured contour information.

In some embodiments, the contouring system may determine the target contouring by using the reference contoured contour information determined based on the reference contouring information.

In some embodiments, the reference contoured contour information may include overall reference contouring information of the first slice image, and the contouring system may further perform a contouring operation on the initial contouring in the second slice image adjacent to the first slice image based on the deformation field and the overall reference contouring information of the first slice image. In this case, the contouring operation may also be called a global deformation mapping operation. The overall reference contouring information of the first slice image may be initial contouring information or modified initial contouring information (e.g., modified contouring information after the user modifies the initial contouring).

The contouring system may determine reference contour pixel points involved in the global deformation mapping operation (such as pixel points whose contour or boundary value is 1 in a reference contoured contour) based on the contouring mask corresponding to the reference contouring information of the first slice image, determine target contour pixel points corresponding to the aforementioned reference contour pixel points in the second slice image based on the deformation field or by using AI recognition, etc., and modify the target pixel points in the second contouring mask, thereby obtaining an updated contouring mask for the second slice image. The target pixel points include target contour pixel points and non-target contour pixel points in the second slice image. Exemplarily, values of the target contour pixels in the second slice image may be set to 1, and values of the non-target contour pixels may be set to 0. Not limited to using the pixcel points, the contour may also be represented by other image features in the slice image, for example, lines, curves, parameters, etc. The contouring system may determine the target contouring of the second slice image according to the updated contouring mask of the second slice image. In this way, the global deformation mapping operation can be completed.

In some embodiments, when a region of interest is contoured in the first image slice and does not contoured in the second image slice, operation 421 may be performed for determining the target contouring of the region of interest. In some embodiments, when a region of interest is contoured in the first image slice and the second image slice, respectively, operation 422 may be performed for determining the target contouring of the region of interest. For example, the target contouring of the region of interest in the second image slice may be determined based on the contouring modification information of the region of interest in the first image slice and the initial contouring of the region of interest in the second image slice.

FIG. 5 is an exemplary flow diagram illustrating a method for registering the first slice image with the second slice image according to some embodiments of the present specification.

In some embodiments, process 500 may be performed by the contouring system. As shown in FIG. 5, the process 500 includes the following steps.

In step 510, for each region of interest (or region of interest along with its associated region), the first slice image is processed based on the initial contouring (or the modified contouring) of the region of interest (or region of interest with its associated region) in the first slice image to obtain a first reference image.

In some embodiments, the contouring system may, based on the initial contouring of the region of interest (or region of interest with its associated region) in the first slice image, perform image enhancement processing on the region of interest (or region of interest with its associated region) in the first slice image to enhance the image quality in the region of interest (or region of interest with its associated region) compared to the region outside the region of interest (or region of interest with its associated region). The image enhancement processing, may be but not limited to, resolution enhancement processing, contrast enhancement processing, color optimization processing, etc. In some embodiments, the contouring system may perform a contrast enhancement processing on the first slice image based on the initial contouring of the region of interest in the first slice image, so as to increase a contrast ratio between a contoured region and an external region in the first slice image. For example, for points inside the contoured region corresponding to the region of interest, their pixel values may be increased (e.g., added or multiplied by a specific pixel value), while for points outside the contoured region, their pixel values may be maintained.

In some embodiments, for the i^{th} region of interest in the first slice image, a corresponding first reference image can be generated based on the following formula (4):

$${T}_{i} ( x ) = ( 1 + \frac{{M}_{{i}^{X}}}{2} ) T ( x ) \qquad Formula ( 4 )$$

where Tᵢ(x) in formula (4) represents the first reference image generated after contrast enhancement is performed on the i^{th} region of interest in the first slice image, and T (x) represents the first slice image. Mᵢx is determined based on the following formula (5):

$${M}_{{i}^{X}} = \{ \begin{matrix}\pm 1 , & x ∈ {O}_{i}^{t} \\ 0 , & else\end{matrix} \qquad Formula ( 5 )$$

where Oᵢ^{t} in formula (5) represents the contoured region corresponding to the i^{th} region of interest in the first slice image.

When the first slice image includes a plurality of regions of interest, a plurality of first reference images corresponding to the plurality of different regions of interest may be generated.

In step 520, for each region of interest (or region of interest with its associated region), the second slice image is processed based on the initial contouring (or the modified contouring) of the region of interest (or region of interest with its associated region) in the second slice image to obtain a second reference image.

In some embodiments, the contouring system may perform image enhancement processing on the region of interest (or region of interest with its associated region) in the second slice image same as the image enhancement processing performed to the first slice image. For example, in some embodiments, similar to the contrast enhancement processing of the first slice image, the contouring system may perform a contrast enhancement processing on the second slice image based on the initial contouring of the region of interest in the second slice image to increase a contrast ratio between a contoured region and an external region in the second slice image.

In some embodiments, for the i^{th} region of interest in the second slice image, a corresponding second reference image can be generated based on the following formula (6):

$${R}_{i} ( x ) = ( 1 + \frac{{N}_{{i}^{X}}}{2} ) R ( x ) \qquad Formula ( 6 )$$

where Rᵢ(x) in formula (6) represents the second reference image generated after contrast enhancement is performed on the ith region of interest, and R (x) represents the second slice image. Nᵢx is determined based on the following formula (7):

$${N}_{{i}^{X}} = \{ \begin{matrix}\pm 1 , & x ∈ {O}_{i}^{r} \\ 0 , & else\end{matrix} \qquad Formula ( 7 )$$

where Oᵢ^{r} in formula (7) represents the contouring of the i^{th} region of interest in the second slice image.

In step 530, a registration objective function is constructed based on the first reference image and the second reference image of each region of interest (or region of interest with its associated region), and the deformation field is determined by solving the registration objective function.

In some embodiments, the contouring system may construct the registration objective function based on the following formula (8):

$$E ( u ) = \frac{{γ}_{0}}{2} {‖ {T}_{0} ( x + u ) - {R}_{0} ( x ) ‖}^{2} + {\sum }_{i = 1}^{k} \frac{{γ}_{i}}{2} {‖ {T}_{i} + ( x + u ) - {R}_{i} ( x ) ‖}^{2} + \frac{α}{2} {‖ {∇}_{u} ‖}^{2} \qquad Formula ( 8 )$$

where E(u) in formula (8) represents the registration objective function, T₀ and R₀ represent the first slice image and the second slice image before the modification operation is performed, respectively, γ₀, γᵢ and α are preset parameter terms, k represents the number of regions of interest, and ∥∇ᵤ∥² is a preset regularization term used to constrain the movement amplitude of the deformation field to be smooth.

In some embodiments, the contouring system may further optimize and solve the registration objective function based on the following formula (9) to obtain the final deformation field u.

u=argminE(u) Formula (9)

where u in formula (9) represents the deformation field, and E (u) represents the above objective function.

In some embodiments of the present specification, the accuracy of subsequent image registration can be improved by performing the contrast enhancement processing on the first slice image and the second slice image. Exceptionally, by adding the existing contourings in the first slice image and the second slice image as prior constraints in the registration algorithm of the first slice image and the second slice image, the registration effect can be optimized, and the precision loss can be reduced when the subsequent layer-by-layer contouring modification is performed based on the deformation field, thereby improving the accuracy of the result of the automated contouring modification.

FIG. 6 is an exemplary flow diagram illustrating one or more rounds of contouring operations according to some embodiments of the present specification.

In some embodiments, the contouring system may perform one or more rounds of contouring operations based on the contouring modification of the second slice image, so as to achieve layer-by-layer mapping of the contouring modification of slice images adjacent to the second slice image.

Each round of contouring operation may be implemented based on process 600. In some embodiments, the process 600 may be performed by the contouring system. As shown in FIG. 6, the process 600 includes the following steps.

In step 610, contouring information in an adjacent slice image of the second slice image is determined based on contouring information in the second slice image in a current round of contouring operation.

The contouring information in the second slice image includes the initial contouring and the target contouring of the second slice image.

In some embodiments, the contouring system may determine contouring modification information of the second slice image based on the contouring information in the second slice image, and perform contouring modification on an initial contouring of the adjacent slice image of the second slice image based on the contouring modification information of the second slice image to determine a target contouring of the adjacent slice image. For example, the contouring system may determine the target contouring of the adjacent slice image of the second slice image based on a deformation field between the second slice image and the adjacent slice image of the second slice image. The adjacent slice image of the second slice image refers to another second slice image whose initial contouring has not been modified and which is adjacent to the foregoing second slice image.

The modification of the initial contouring of the adjacent slice image based on the contouring modification information of the second slice image is similar to the modification of the initial contouring of the second slice image based on the contouring modification information of the first slice image as shown in FIG. 3 (such as step 330), and will not be repeated here.

It should be noted that each round of contouring operation may be implemented based on steps 410 to 420 (such as step 421 or step 422) shown in FIG. 4.

In step 620, it is determined whether a termination condition is met.

The termination condition is used to determine whether one or more rounds of contouring operations need to be stopped. For example, the termination condition may be that contouring modifications of all slice images containing the region of interest in the slice image sequence are completed. For example, the termination condition may be manually terminated by the user, for example by entering a termination instruction.

In step 631, in response to the termination condition being not met, the adjacent slice image of the second slice image is designated as a second slice image in a next round of contouring operation.

In some embodiments, as shown in FIG. 6, in a case that the termination condition is not met, the contouring system may designate the adjacent slice image of the second slice image of the current contouring operation as the second slice image in the next contouring operation, and continue to return to step 610 to perform the next contouring operation.

In step 632, in response to the termination condition being met, one or more rounds of contouring operations are stopped.

In other words, in a case that the termination condition is met, the contouring modifications for the plurality of slice images (such as part or all of the second slice images) can be stopped.

In some embodiments, after the contouring operations are stopped, a notification may be generated to alert the user. The notification may be in the form of text, sound, etc., or a combination thereof.

In some embodiments, the contouring system may determine a difference between the initial contouring and the target contouring in the adjacent slice image of the second slice image; determine, in response to the difference being greater than a preset difference threshold, that the termination condition is not met; and determine, in response to the difference being less than or equal to the preset difference threshold, that the termination condition is met.

The difference between the initial contouring in the adjacent slice image and the target contouring in the adjacent slice image may be referred to as a contouring difference, which may reflect the magnitude of the modification amount from the initial contouring to the target contouring in the adjacent slice image. In some embodiments, the magnitude of the modification amount may be determined based on a mask difference between a target contouring mask corresponding to the target contouring of the adjacent slice image and an initial contouring mask corresponding to the initial contouring of the adjacent slice image.

It is understandable that the target contouring is obtained by modifying the initial contouring. When the mask difference is larger, it is indicated that the difference between the initial contouring and the target contouring is larger, and the required modification amount is larger. In this case, the contouring operation needs to be continued. On the contrary, when the mask difference is smaller, it is indicated that the initial contouring is closer to the target contouring, the required modification amount is smaller, and the contouring operation is more suitable to stop. It can be understood that a balance between an effect of the target contouring and the required modification amount can be evaluated through the preset difference threshold, so as to reduce the unnecessary consumption of computing resources of the system.

In some embodiments, the contouring difference is also related to modified contour points of the contouring modification. The more contours are modified, the greater the modification amount required. In this case, the contouring operation needs to be continued. The number of the modified contour points may be determined based on the similarity degree between the initial contouring and the target contouring of the adjacent slice image.

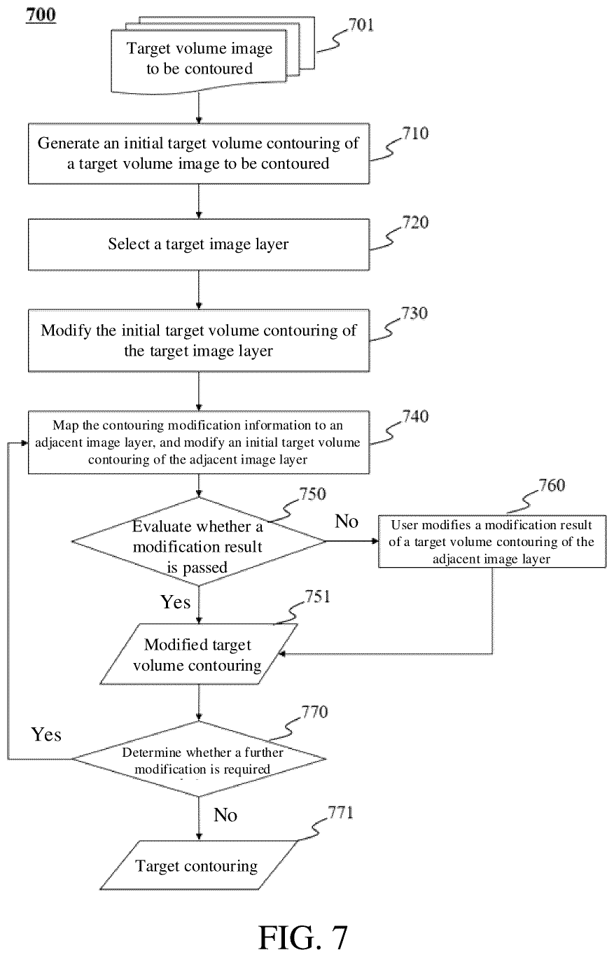

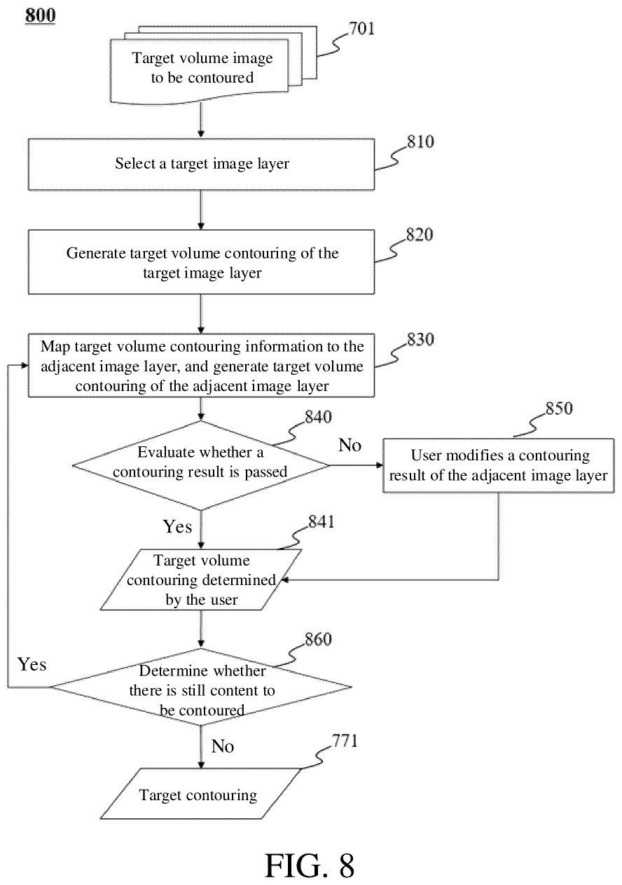

In some embodiments, the termination condition may include a contouring score being above a threshold score. The contouring score may be determined based on the contouring difference. The smaller the contouring difference, the higher the contouring score, otherwise, the lower the contouring score. The threshold score may be pre-determined (e.g., determined based on experience). In a case that the contouring score is greater than the threshold score, the above multiple rounds of contouring operations are stopped. It should be noted that whether to terminate multiple rounds of contouring operations can also be directly determined by the user (such as a physician). For more details, FIGS. 7 and 8 and the corresponding descriptions are referred.

In some embodiments, after the contouring operations are stopped, a three-dimensional reconstruction may be performed on the slice image sequence obtained after the contouring operations. Exemplarily, the contouring system may reconstruct the contours of the target volume and the OARs finally obtained in the first slice image and the second slice images after the contouring modifications are completed in a three-dimensional space, so as to generate a closed three-dimensional curved surface, which can be presented in the form of a three-dimensional model on the terminal device for the user to view or confirm. It should be noted that the user may also fine-tune a contouring result of one or more slice images individually according to the viewed or confirmed result.

In some embodiments, at least one region of interest includes target volume to be treated. As shown in FIG. 6, in the case that the termination condition is met, the process 600 may further include step 640.

In step 640, a treatment plan for the target volume is generated based on the first slice image and the second slice images after the contouring operations.

In some embodiments, the contouring system may generate the treatment plan for the target volume based on the target contourings in the slice images that has been performed with the contouring modifications and/or the initial contourings in the slice images that has not been performed with contouring modifications.

The treatment plan for the target volume refers to a plan for treating the target volume (such as radiotherapy). In some embodiments, the contouring system may determine the treatment plan for the target volume based on relevant information of the target volume and relevant information of the OARs. For example, the dosage, duration of treatment, etc. of the treatment plan are determined based on the location, size, and dimensions of the target volume in the contoured slice image, as well as the type and location of the OAR.

In some embodiments of the present specification, the treatment plan for the target volume can be automatically determined through the completed contouring result, so as to better assist doctors in treating patients, thereby improving user experience while reducing the time and energy consumption of the entire treatment process.

In some embodiments of the present specification, the plurality of slice images can be automatically contoured and modified layer-by-layer through one or more rounds of contouring operations, greatly reducing the workload and time of doctors in the contouring modification.

FIG. 7 is an exemplary flow diagram illustrating another image contouring method according to some embodiments of the present specification.

In some embodiments, as shown in FIG. 7, process 700 may include the following steps.

In step 710, an initial target volume contouring of a target volume image to be contoured is generated.

The target volume image 701 to be contoured may be one or more slice images of the three-dimensional medical image. The initial target volume contouring refers to an initial contouring of a region of interest in the target volume image to be contoured. In some embodiments, step 710 may be performed by the contouring system. For example, the contouring system may contour the target volume image 701 to be contoured based on a preset segmentation algorithm to generate the initial target volume contouring. For relevant information about the slice image and the initial contouring, step 320 and the corresponding description are referred, which will not be repeated here.

In step 720, a target image layer is selected. In some embodiments, step 720 may be performed by the contouring system. For example, the contouring system may select a slice image in the target volume image 701 to be contoured as the target image layer. In some embodiments, the target image layer may also be selected or specified by a user (e.g., a physician).

In step 730, the initial target volume contouring of the target image layer is modified. In some embodiments, step 730 may be performed by the contouring system. In some embodiments, the contouring system may modify the initial target volume contouring of the target image layer by obtaining an adjustment instruction (such as an input instruction generated by a keyboard, a mouse, etc.) for the initial target volume contouring from the user, and obtain contouring modification information. The modified target image layer can refer to a first slice image. For relevant information about the first slice image and the contouring modification information, the description in step 330 is referred.

In step 740, the contouring modification information is mapped to an adjacent image layer, and an initial target volume contouring of the adjacent image layer is modified.

In some embodiments, step 740 may be performed by the contouring system. The adjacent image layer refers to one or more second slice images. For example, the adjacent image layer includes a second slice image adjacent to the target image layer (the first slice image), and other second slice images adjacent to the second slice image.

In some embodiments, the contouring system may register the first slice image with the second slice image to determine a deformation field between the first slice image and the second slice image, which can be achieved based on the method described in step 410. Further, the contouring system may perform a local deformation mapping operation on an initial contouring in the second slice image based on the deformation field and the contouring modification information, so as to map the contouring modification information to the adjacent image layer, and modify the initial target volume contouring of the adjacent image layer, which can be achieved based on the method described in step 421, and will not be repeated here.

In some embodiments, step 740 includes one or more rounds of contouring operations (local deformation mapping operations), so as to modify the initial contouring of one or more second slice images in the target volume image 701 to be contoured. For relevant information about one or more rounds of contouring operations, FIG. 6 and the corresponding description are referred.

In step 750, it is evaluated whether a modification result is passed.

The modification result refers to a target volume contouring of the second slice image obtained after the contouring system performs one or more rounds of contouring operations (local deformation mapping operations). In some embodiments, the user may review the modification result on a display device, and determine whether the modification result is passed based on experience.

In a case that the modification result is passed, a modified target volume contouring 751 corresponding to one or more slice images in the target volume image 701 to be contoured is obtained.

In step 760, the user modifies a modification result of a target volume contouring of the adjacent image layer.

In a case that the modification result is not passed, the user may also modify (such as fine-tuning the contour of the target volume contouring corresponding to the modification result) the modification result corresponding to the target volume contouring in the adjacent image layer (such as one or more second slice images), so as to obtain the modified target volume contouring 751.

In step 770, it is determined whether a further modification is required.

In some embodiments, the user may also evaluate the modified target volume contouring 751 to determine whether the further modification is required. For example, the user may also evaluate an overall contouring effect of the target volume image 701 to be contoured (such as evaluate whether the target volume contouring among the plurality of adjacent slice images meets the requirements of smoothness and continuity) to determine whether the further modification is required.

In a case that no further modification is required, a target contouring 771 of the target volume image 701 to be contoured is obtained. In a case that the further modification is required, the user may perform an operation similar to step 730, and the contouring system executes the above step 740 to finally obtain the target contouring 771.

FIG. 8 is an exemplary flow diagram illustrating yet another image contouring method according to some embodiments of the present specification.

In some embodiments, as shown in FIG. 8, process 800 may include the following steps.

In step 810, a target image layer is selected.

Step 810 is similar to step 720, and the selection of the target image layer in the target volume image 701 to be contoured may be implemented by the contouring system or by the user. For relevant information, step 720 and the corresponding description are referred.

In step 820, a target volume contouring of the target image layer is generated.

In some embodiments, step 820 may be performed by the contouring system. For example, the contouring system may generate the target volume contouring of the target image layer based on a preset segmentation algorithm. Step 820 can be implemented by the user contouring according to information (such as location information of the region of interest, etc.) of the target image.

In step 830, target volume contouring information is mapped to the adjacent image layer, and target volume contouring of the adjacent image layer is generated.

In some embodiments, step 830 may be performed by the contouring system. The target volume contouring information refers to reference contoured contour information corresponding to the target volume contouring of the target image layer (the first slice image). In some embodiments, the contouring system may perform a global deformation mapping operation based on the target contouring information to generate the target volume contouring of the adjacent image layer of the target image layer. For relevant information, step 422 and the corresponding description are referred.

In some embodiments, step 830 includes one or more rounds of contouring operations (global deformation mapping operations), so as to generate the target volume contouring of one or more second slice images in the target volume image 701 to be contoured. For relevant information about one or more rounds of contouring operations, FIG. 6 and the corresponding description are referred.

In step 840, it is evaluated whether a contouring result is passed.

The contouring result refers to a target volume contouring of the second slice image obtained after the contouring system performs one or more rounds of contouring operations (global deformation mapping operations). In some embodiments, the user may review the contouring result on a display device, and determine whether the contouring result is passed based on experience.

In a case that the contouring result is passed, a target volume contouring 841 determined by the user corresponding to one or more slice images in the target volume image 701 to be contoured is obtained.

In step 850, the user modifies a contouring result of the adjacent image layer.

In a case that the contouring result is not passed, the user may modify (such as fine-tuning the contour of the target volume contouring corresponding to the contouring result) the contouring result corresponding to the target volume contouring in the adjacent image layer (such as one or more second slice images), so as to obtain the target volume contouring 841 determined by the user.

In step 860, it is determined whether there is still content to be contoured.

In some embodiments, the user may evaluate whether there is still content that needs to be contoured (such as the contourings of other regions of interest) in the first slice image, so as to determine whether there is still content that needs to be contoured.

In a case that there is nothing to be contoured, a target contouring 771 of the target volume image 701 to be contoured is obtained. In a case that there is still content to be contoured, the user may perform an operation similar to step 830, and the contouring system executes the above step 840 to finally obtain the target contouring 771.

In some embodiments of the present specification (such as the embodiments of FIG. 7 and FIG. 8), based on the contouring operation of the system, and combined with user evaluation and fine-tuning processing, it is ensured that the target contouring of the target volume image to be contoured can meet actual clinical requirements, thereby treating patients more accurately.

It should be noted that the above description related to the processes is only for example and explanation, and does not limit the scope of application of the present specification. For those skilled in the art, various modifications and changes can be made to the process under the guidance of the present specification. However, these modifications and changes are still within the scope of the present specification.

The basic concepts have been described above. Obviously, for those skilled in the art, the above detailed disclosure is only an example and does not constitute a limitation of the present specification. Although not explicitly described herein, those skilled in the art may make various modifications, improvements and corrections to the present specification. Such modifications, improvements and corrections are suggested in the present specification, so such modifications, improvements and corrections still fall within the spirit and scope of the exemplary embodiments of the present specification.

Meanwhile, the present specification uses specific words to describe the embodiments of this specification. The terms “one embodiment”, “an embodiment”, and/or “some embodiments” refer to a particular feature, structure, or characteristic related to at least one embodiment of this specification. Therefore, it should be emphasized and noted that “an embodiment”, or “an embodiment”, or “an alternative embodiment” mentioned twice or more times in different places in this specification does not necessarily refer to the same embodiment. Furthermore, certain features, structures or characteristics in one or more embodiments of this specification may be appropriately combined.

In addition, unless explicitly stated in the claims, the order of the processing elements and sequences, the use of alphanumeric characters, or the use of other names described in this specification are not intended to limit the order of the processes and methods of this specification. Although the above disclosure discusses some of the invention embodiments currently considered useful through various examples, it should be understood that such details are only for illustrative purposes, and the appended claims are not limited to the disclosed embodiments. On the contrary, the claims are intended to cover all modifications and equivalent combinations that are consistent with the essence and scope of the embodiments of this specification. For example, although the system components described above can be implemented by hardware devices, they can also be implemented by software-only solutions, such as installing the described system on an existing server or mobile device.

Similarly, it should be noted that in order to simplify the expressions disclosed in this specification to facilitate the understanding of one or more embodiments of the invention, various features are sometimes combined into one embodiment, figure, or description thereof in the above description of the embodiments of this specification. This method of disclosure, however, does not imply that the subject matter of the specification requires more features than are expressly recited in the claims. In fact, embodiments may have less than all of the features of a single foregoing disclosed embodiment.

In some embodiments, numbers are used to describe the quantities of components and attributes. It should be understood that such numbers used in describing the embodiments are modified by modifiers such as “about”, “approximately”, or “substantially” in some examples. Unless otherwise stated, “about”, “approximately”, or “substantially” indicates that the stated number allows for a variation of ±20%. Accordingly, in some embodiments, numerical parameters used in the specification and claims are approximations that may vary depending upon the desired characteristics of a particular embodiment. In some embodiments, the numerical parameters should take into account the specified number of significant digits and adopt the general method of preserving the number of digits. Although the numerical ranges and parameters used to identify the breadth of their ranges in some embodiments of this specification are approximate, in specific embodiments, such numerical values are set as accurately as possible within the feasible range.

For each patent, patent application, patent application publication, and other materials, such as articles, books, specifications, publications, documents, etc., cited in this specification, the entire contents are hereby incorporated into this specification by reference. Application history documents that are inconsistent with or conflicting with the contents of this specification are excluded, as are documents (currently or later attached to this specification) that limit the broadest scope of the claims of this specification. It should be noted that if there is any inconsistency or conflict between the descriptions, definitions, and/or usage of terms in the supplementary materials of this specification and the contents described in this specification, the descriptions, definitions, and/or usage of terms in this specification shall prevail.

Finally, it should be understood that the embodiments described in the present specification are only used to illustrate the principles of the embodiments of the present specification. Other variations are also possible and fall within the scope of the present specification. Therefore, by way of example and not limitation, alternative configurations of the embodiments of the present specification may be considered consistent with the teachings of the present specification. Accordingly, the embodiments of the present specification are not limited to the embodiments explicitly introduced and described in the present specification.

What is claimed is:

1. An image contouring method, comprising:
obtaining a plurality of slice images;
obtaining reference contouring information in a first slice image among the plurality of slice images; and
determining, based on the reference contouring information, a target contouring in a second slice image among the plurality of slice images.

2. The method according to claim 1, wherein the plurality of slice images are arranged in sequence, and the second slice image is adjacent to the first slice image.

3. The method according to claim 1, wherein the reference contouring information comprises a prior constraint for defining the target contouring.

4. The method according to claim 1, wherein the reference contouring information comprises contouring information for at least one region of interest.

5. The method according to claim 4, wherein the reference contouring information further comprises contouring information for an associated region around the at least one region of interest.

6. The method according to claim 1, wherein obtaining the reference contouring information in the first slice image among the plurality of slice images comprises:
generating initial contouring information in the first slice image among the plurality of slice images; the initial contouring information containing an initial contouring of at least one region of interest in the first slice image;
obtaining, based on the initial contouring information, the reference contouring information in the first slice image.

7. The method according to claim 6, wherein the initial contouring information is determined based on manual contouring, or the initial contouring information is determined based on a preset segmentation algorithm or Artificial Intelligence (AI).

8. The method according to claim 1, wherein determining, based on the reference contouring information, the target contouring in the second slice image among the plurality of slice images comprises:
modifying, by using contouring modification information determined based on the reference contouring information, an initial contouring in the second slice image to determine the target contouring.

9. The method according to claim 8, wherein the contouring modification information reflects a difference between the initial contouring and a modified contouring of the first slice image.

10. The method according to claim 9, wherein the initial contouring is represented by a first contouring mask and the modified contouring is represented by a second contouring mask; and
the contouring modification information includes a contouring modification mask determined by obtaining a difference between the first contouring mask and the second contouring mask.

11. The method according to claim 8, wherein modifying, by using the contouring modification information determined based on the reference contouring information, the initial contouring in the second slice image comprises:
registering the first slice image with the second slice image to determine a deformation field between the first slice image and the second slice image; and
modifying, based on the deformation field and the contouring modification information, the initial contouring.

12. The method according to claim 11, wherein the registering the first slice image with the second slice image to determine a deformation field between the first slice image and the second slice image includes:
determining the deformation field between the first slice image and the second slice image based on the initial contouring of the second slice image and the initial contouring or a modified contouring of the first image slice.

13. The method according to claim 8, wherein modifying, by using contouring modification information determined based on the reference contouring information, an initial contouring in the second slice image to determine the target contouring comprises:
determining modified part of contouring in the first slice image as the contouring modification information of the first slice image; and
modifying corresponding part of the initial contouring in the second slice image corresponding to the modified part to determine the target contouring of the second slice image.

14. The method according to claim 8, wherein modifying, by using contouring modification information determined based on the reference contouring information, an initial contouring in the second slice image to determine the target contouring comprises:
determining modified pixel points based on the contouring modification information of the first slice image; and
modifying corresponding pixel points in the second slice image corresponding to the modified pixel points to determine the target contouring of the second slice image.

15. The method according to claim 1, wherein determining, based on the reference contouring information, the target contouring in the second slice image among the plurality of slice images comprises:
determining, by using reference contoured contour information determined based on the reference contouring information, the target contouring.

16. The method according to claim 13, wherein determining, by using the reference contoured contour information determined based on the reference contouring information, the target contouring comprises:
registering the first slice image with the second slice image to determine a deformation field between the first slice image and the second slice image; and
determining, based on the deformation field and the reference contoured contour information, the target contouring.

17. The method according to claim 15, wherein determining, by using reference contoured contour information determined based on the reference contouring information, the target contouring comprises:
determining based on the reference contoured contour information of the first slice image reference, reference contour pixel points involved in a global deformation mapping operation;
determining target contour pixel points corresponding to the reference contour pixel points in the second slice image, based on the deformation field; and
modifying the target contour pixel points in the second slice image to determine the target contouring of the second slice image.

18. The method according to claim 1, wherein the method further comprises:
performing one or more rounds of contouring operations, each round of contouring operation comprising:
determining, based on contouring information in the second slice image in a current round of contouring operation, contouring information in an adjacent slice image of the second slice image; the contouring information comprising an initial contouring or a target contouring;
determining whether a termination condition is met; and
designating, in response to the termination condition being not met, the adjacent slice image as a second slice image in a next round of contouring operation, and performing the next round of contouring operation; or
stopping, in response to the termination condition being met, the one or more rounds of contouring operations.

19. An image contouring system, comprising a storage device and a processing device, the storage device being stored with computer instructions, which, when executed by the processing device, performs steps to:
obtain a plurality of slice images;
obtain reference contouring information in a first slice image among the plurality of slice images; and
determine, based on the reference contouring information, a target contouring in a second slice image among the plurality of slice images.

20. A non-transitory computer-readable storage medium storing instructions therein, wherein the instructions, when executed by a processor, cause the processor to implement steps to:
obtain a plurality of slice images;
obtain reference contouring information in a first slice image among the plurality of slice images; and
determine, based on the reference contouring information, a target contouring in a second slice image among the plurality of slice images.
